(19) United States
(12) Patent Application Publication
(10) Pub. No.: US 2003/0230478 A1
(43) Pub. Date: Dec. 18, 2003

(54) Process for the preparation of 1,4 - dihydropyridines and novel 1,4-dihydropyridines useful as therapeutic agents

(75) Inventors: Neeraj Mahendroo, Jannu (IN); Ravi Kumar Khajuria, Jannu (IN); Kasturi Lal Bedi, Jannu (IN); Kanaya Lal Dhar, Jannu (IN); Vijay Kumar Kapoor, Chandigarh (IN)

Correspondence Address: LADAS & PARRY, 26 WEST 61ST STREET, NEW YORK, NY, 10023, US

(73) Assignee: COUNCIL OF SCIENTIFIC AND INDUSTRIAL RESEARCH

(21) Appl. No.: 10/393,373

(22) Filed: Mar. 20, 2003

(30) Foreign Application Priority Data
May 29, 2002 (WO) PCT/IN02/00125

(51) Int. Cl. C07D 211/90

(52) U.S. Cl. 204/157.71; 546/318

(57) ABSTRACT

The present invention provides a process for the preparation of 1,4-dihydropyridines of the formula 1

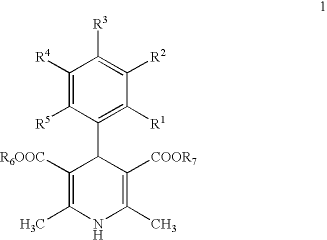

wherein R₁ is H, NO₂, Cl, OAc, OH, R₂ is H, NO₂, Cl, —O—CH₂—O—, OMe, OAc, OEt, OH, R₃ is H, NO₂, Cl, N(Me)₂, —O—CH₂—O—, OMe, OAc, OH, R₄ is H, OMe, OAc, OH, R₅ is H, Cl, I, and R₆ and R₇ are either methyl, ethyl or both by preparing a mixture of an aromatic aldehyde, alkyl acetoacetate and a source of ammonia, adsorbing the prepared mixture on adsorbent till adsorbent becomes free flowing, heating the material so obtained under microwave irradiation, cooling the reaction mixture and recovering the compound of formula I. The present invention also relates to novel 1,4-dihydropyridines.

### FIELD OF THE INVENTION

The present invention relates to a process for the preparation of 1,4-dihydropyridines useful as therapeutic agents and to novel compounds obtained thereby. The present invention particularly relates to 4-aryl-2,6-dimethyl-3,5-dicarboalkoxy-1,4-dihydropyridines such as 4-aryl-2,6-dimethyl-3,5-dicarbomethoxy-1,4-dihydropyridines, 4-aryl-2,6-dimethyl-3,5-dicarboethoxy-1,4-dihydropyridines, and 4-aryl-2,6-dimethyl-3-carboethoxy-5-carbomethoxy1,4-dihydropyridines as racemates with the aid of microwave irradiation. These dihydropyridines are potential cardiovascular agents.

### BACKGROUND OF THE INVENTION

In recent times there has been a tremendous interest in microwave mediated organic synthesis. The main advantages are, shorter reaction time, cleaner reaction, better yields, ease of work up after reaction, reduction in thermal degradation, better selectivity and environment friendly conditions (Abramovitch, R. A., Tetrahedron Lett., 32, 1991,5271. Bose, A. K.; M. S.; Ghosh, M.; Shah, M.; Raju, V. S.; Bari, S. S.; Newaz, S. N.; Banik, B. K.; Chaudhary, A. G.; Barkat, K. J., J. Org. Chem., 56, 1991, 6968. Caddick, S., Tetrahedron, 51, 1995, 10403. Staruss, C. R.; Trainor, R. W., Aust. J. Chem., 48, 1995, 1665. Bose, A. K.; Jayarraman, M.; Okawa, A.; Barri, S. S.; Robb, E. W.; Manhas, M. S., Tetrahedron Lett., 37, 1996, 6989. Verma, R. S.; Dahia, R.; Saini R. K., Tetrahedron Lett., 38, 1997, 7029.). The reactions can be carried out from a few milligrams to 500 grams quantities in a simple household microwave oven (Banik, B. K.; Manhas, M. S.; Kaulza Z.; Barakat K. J.; Bose A. K., Tetrahedron Lett., 33, 1992, 3603). Microwave ovens can range from simple household multimode ovens to large-scale batch as well as continuous multimode ovens. In batch closed reactor, vessels or turntables having capacity to contain a number of reaction vessels, have been applied. Specifically in food industry large-scale (continuous mode) ovens are used frequently (Galema, S. A., Chem. Soc. Rev., 26, 1997. Staruss, C. R.; Trainor, R. W., Aust. J. Chem., 48, 1995, 1665). Thus it is possible to scale up reactions to industrial scale. Calcium entry blockers, 1,4-dihydropyridines have rapidly emerged as an important class of nitrogen heterocycles used for the management of cardiovascular diseases. These compounds have occupied an important position as therapeutic agents among various types of calcium entry blockers. Interest in dihydropyridines chemistry can be traced back to Co-enzyme reduced nicotinamide adenine dinucleotide and the unique ability of this compound in biological systems to reduce unsaturated functionalities (carbonyls, conjugated olefins etc.). Thus, a considerable portion of today's efforts in dihydropyridine chemistry is extended to synthizing NADH (2) mimics, exploring the reactions and mechanisms of these compounds and utilizing these compounds in a variety of synthetic reactions.

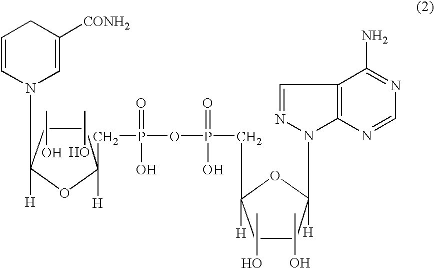

The first generation dihydropyridines are one possessing identical ester functions at position 3 and 5 in the dihydropyridine ring. The second generation of dihydropyridine (DHP) development candidates have greater potency and are all chiral owing to non-identical ester functions (Meyer, H; Bossert, F.; Wehiger, E.; Stoepel, K.; Vater, W., Arzneim.-Forsch./Drug Res., 30, 1981, 407.) and can exist in enantiomeric forms differing in absolute configuration at C-4 (Towart, R.; Wehinger, E.; Meyer, H.; Schmiedbergs, N., Arch. Pharmacol., 317,1981, 183.). Felodipine (27) is one of these DHP candidates, which have non-identical ester function and is characterized by pronounced peripheral vasodilation, so it appears to be particularly interesting for therapy of hypertension (Ek, B.; Ahnoft, M.; Norlander, H. H.; Jung, B. L., Arch. Pharmacol., 313, 1980, Supl. R37.).

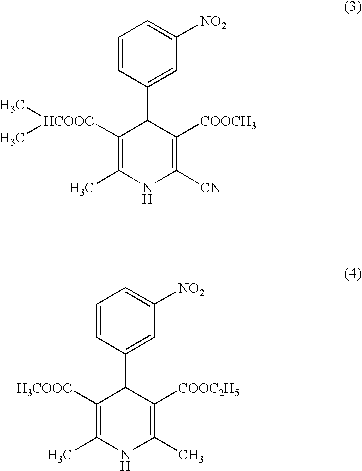

Another DHP which has non-identical ester functionalities is nivaldipine (3) which is also substituted at the position-2 by cyano group in place of customary methyl group (Jully, S. R.; Hardmann, H. F.; Gross, G. H., J. Pharmacol. Exp. Ther., 217, 1981, 20.). Nitrendipine (4) another 1,4-dihydropyridine derivative with mixed ester functionalities is being marketed as antihypertensive drug which has longer duration of action than nifedipine (5). A marked therapeutic response is obtained on administration of nitrendipine (4) in hypertensive patients with coronary artery diseases who respond inadequately to β-blockers (Scriabine, A.; Vanov, S.; Deek, K. Eds., Nitrendipine, Urban & Schwartzenberg, Tokyo, 1983.).

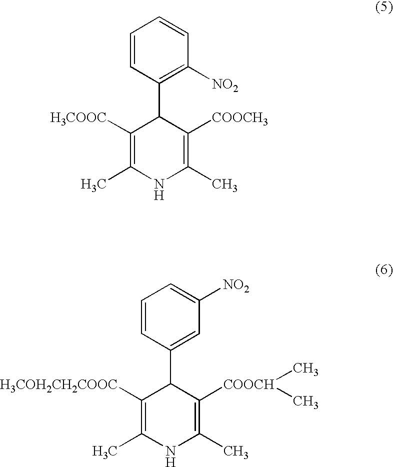

Nimodipine (6) due to its dilative action on spasm of cerebral vessels, has also become a drug of choice in patients with subarchanoid hemorrhage (Betz, E.; Deek, K.; Hoffmeister, F. Eds., Nimodipine, Pharmacological and Clinical Properties, F. K. Schattener Verlag, Stutgart, New York, 1985.). Nimodipine crosses the blood brain barrier and elicits some direct psyhotropic activity and also dilates cerbral vessels to a greater extent (Baldwin, J. J.; Hirschmann, R.; Engekhardt, E. L.; Pinticello, G. S.; Sweet, C. S.; Scribine, A., J. Med. Chem., 24, 1981, 628.).

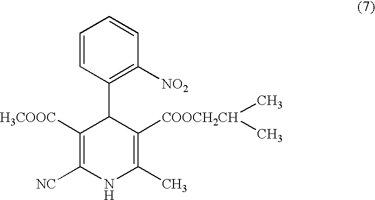

Nisoldipine i.e. Bay k 5552 (7) is one of the most potent blockers of voltage dependent Ca²⁺ channels and is characterized by its predominating effects on the coronary and peripheral blood vessels (Kazda, S.; Garthoff, B.; Ramsch, K. D.; Schluter, G., New Drugs of annual, Cardiovascular Drugs 1, A. Scribane Ed., Raven Press, New York, 243, 1983. Itoh, T.; Kannura, Y.; Kariyama, H.; Suzuki, H., Br. J. Pharmacol., 83, 1984, 243.).

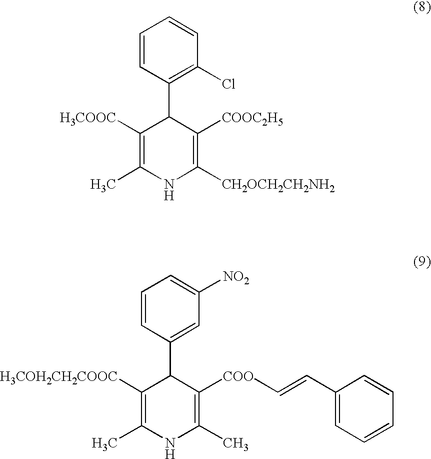

Amlodipine (8) another DHP with non-identical ester functionalites and a basic side chain at position 2 is a long acting dihydropyridine with a half life of 30 hrs in dogs. Bulk of activity resides in (−) isomer of (8) which has shown extensive potential as antihypertensive and antianginal drug (Arrowsmith, J. E., Campbell S. F.; Cross, P. E. Stubbs, J. K.; Burges, R. A.; Gardiner, D. G.; Blackburn, K. J., J. Med. Chem., 29, 1986, 1696. Alker, D.; Campbell, S. F.; Cross, P. E., J. Med. Chem., 34, 1991, 19.).

Structural modifications among dihydropyridines continue to be concentrated on the ester and aryl substituents with the aim of discovering examples having slow onset and long duration of action. Results with FRC-8653 (9) are encouraging. In spontaneous hypertensive rats, this compound exhibits an antihypertensive effect with slow onset and a longer duration of action (Iida, H.; Fujiyoshi T.; Ikeda, K.; Hosoro, M.; Yanura, M.; Kase, N.; Sekive, A.; Uematsu, T., Japan. J. Pharmacol., 43, 1987, 296.).

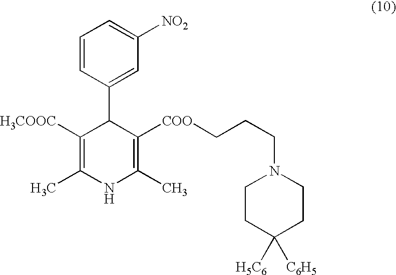

Similar long lasting effects were also observed in dogs with B844-39 (10) (Fisher, G.; Krumple, G.; Mayer, N.; Schneider, W.; Raberger, G. J., Cardiovasc. Pharmacol., 10, 1987, 268.).

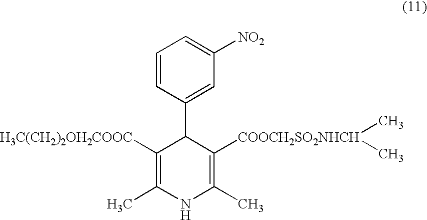

More extensive structural modifications of ester moiety are found in RO18-3017 (11) (Holik M.; Osterrieder, W., Br. J. Pharmac., 91, 1987, 61.).

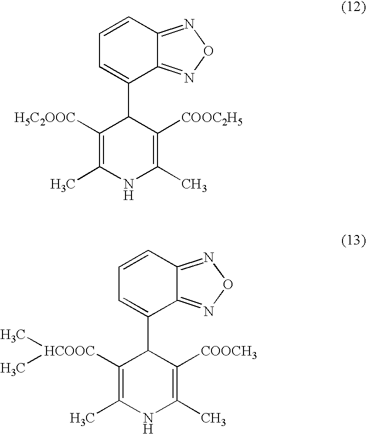

Compound PN 200-110 (13) is more potent than (12) due to mixed ester functionalities (Hof, R. P.; Schweinitzer, M. E.; Neumann, P., Br. J. Pharmacol., 73, 1981,196.)

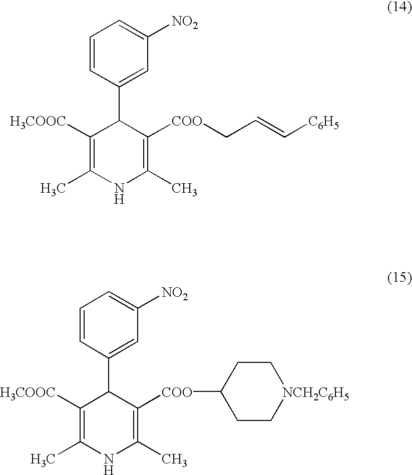

FRC-8411 (14) shows good hypotensive and antianginal activites (Yamaura, T.; Kase, N.; Kita, H.; Uematsu, T., Arzeneim.-Forsch./Drug Res., 36, 1986, 29.). Compound YM-09730 (15) shows greatest coronary vasodilating activity (Tamnzawa, K.; Arima, H.; Kojima, T.; Tsomura, Y.; Okeda, M.; Fujita, S.; Furuya, T.; Takeneda, T.; Inagaski O.; Terai, M., J. Med. Chem., 29, 1986, 2504.).

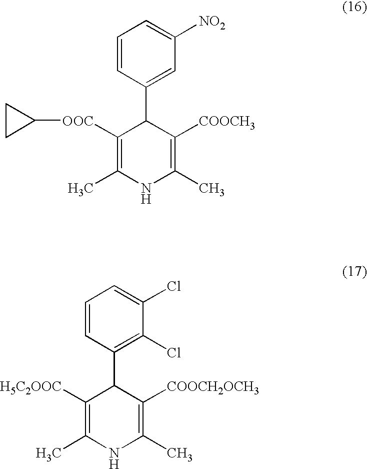

Compounds (16) and (17) have been reported to have activity similar to felodipine (27) (shown to be 1000 folds more potent than nifedipine (5)) (Ohno, S.; Kamatsu, O.; Miznokoshi, K.; Jahihara K.; Nakamura, Y.; Marighima, I.; Sumuta, K., J. Pharm. Dyn., 7,1984,5).

Nigulidipine (18) developed orginally as a long acting calcium channel blocker, also increases the opening probability of Ca²⁺ activated K⁺ channels and may be example of a compound acting in opposite fashion on two distinct ion channels (Robertson, D. W.; Steinberg, M. I., J. Med. Chem., 33, 1990, 1).

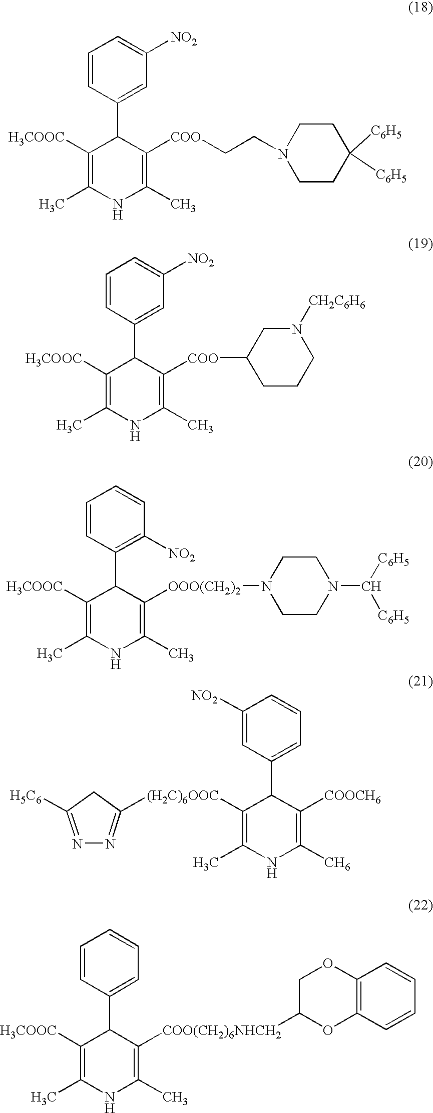

New dihydropyridines, benidipine (19), manidipine (20), CV-159 (21) and P-0285 (22), have been found to be more potent and have specific vascular effects than prototypes m class of compounds related to nifedipine (5). They have been reported to have slow onset and long duration of action in animals without cardiodepressant effect (characteristic of other dihydropyridines).

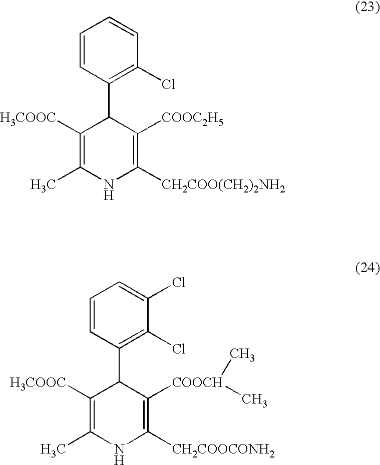

Compounds like NB-818 (23), are more potent and have longer duration of action in vivo than agents like nifedipine (5). Compound (24) has been reported to increase cerebral cortical blood flow and improve memory in certain models (Naurse, T.; Kiozumi, Y., Japan. J. Pharmacol., 46(Suppl.),1988, 75. Nichikibe, A.; Nakajuma, A., Life Sciences, 43, 1988, 1715)

Available literature discloses number of methods for the preparation of 1,4-dihydropyridines involving condensation of various substituted aldehydes with methylacetoacetate or ethylacetoacetate in presence of ammonia using methanol or ethanol as solvent. However, so far no attempt has been made to provide clean, safe, time-saving, environment friendly and an inexpensive method for the preparation of 1,4-dihydropyridines. This method can only be provided if the reaction is carried out under the influence of microwave irradiation. There is only one method in the literature for the preparation of 1,4-dihydropyridines with the help of microwave irradiations where the ammonia, alkylacetoacetate and aldehyde with ethanol as solvent have been used for the preparation of 1,4-dihydropyridines (Alajarin, R.; Vaquero, J. J.; Garcia, Navio, J. L.; Alavarez-Builla, J., Synlett., 1992, 297.). In the literature no method is available where the preparation of 1,4-dihydropyridines have been carried out under dry conditions on a solid support. Methods where solvents such as ethanol or methanol are used for preparation of various compounds under influence of microwave irradiation suffer from one or the other drawbacks such as inflammability due to switching on and off of the magnetron of microwave oven to control the power out put. There is no method in literature where 4-aryl-2,6-dimethyl-3-carboethoxy-5-carbomethoxy-1,4-dihydropyridine have been prepared in a single step.

Clinical usefulness of nifedipine (5), a prototype of dihydropyridines, in the management of cardiovascular diseases, (Fleckenstein, Von, A.; Trithart, H.; Doring, H. J.; Byon, K. X., Arzneim.-Forsch./Drug Res., 22, 1972, 1) stimulated extensive research in this area leading to the discovery of a large number of 1,4-dihydropyridines which have been found to be more potent than the nifedipine (5). For example, discovery of nicardipine (30) a cerebrovasodilating agent has been a subject of great interest since the increase of cerebrovascular diseases in recent years. 1,4-dihydropyridines have been found to be of utmost importance in the biological systems (Brjuce, T. C.; Benkovic, S. J., “Biorganic Mechanisms” W. A. Benjamin, New York, N.Y., 2, 1966, 301. Florkin, M.; Stotz, E. H. Ed. “Comprehensive Biochemistry” 14, Elsevier Amsterdam, 1996) and the superior calcium antagonistic dihydropyridines have initiated the development of large number of analogues as primary antianginal/anti-hypertensive agents.

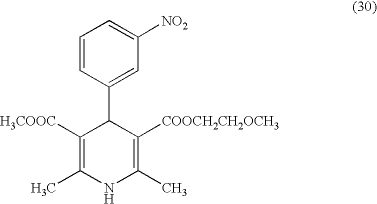

Dihydropyridines chemistry began in 1882 when Hantzsch (Hantzsch, A., Justus Liebigs. Ann. Chem., 1, 1882, 15) published the synthesis which bears his name. In the subsequent 50 years, modifications of the original synthesis were developed and some reactions of dihydropyridines were studied. Dihydropyridines are readily convertible to pyridines and are important intermediates in the synthesis of latter. Detailed survey of synthetic reactions covering the literature (Eisner, U.; Kuthan J., Chem. Rev., 72, 1972, 1) has been published. Dihydropyridines also play an important role as intermediates in reactions of pyridines e.g. in nucleophilic substitutions (Abramovitch, R. A; Saha, J. G., Advan. Heterocycl. Chem., 6,1966, 224) and reductions (Lyle, R. E.; Anderson, P. S., Advan. J. Heterocycl. Chem., 6, 1966, 45) as well as acylations in the presence of pyridine (Doering, W. Von; McEdwen, W. E., J. Amer. Chem. Soc., 73, 1951,2104).

Dihydropyridines are of utmost importance in biological systems especially NADH (2) which is involved in the biological redox reactions. Pharmacological properties of dihydropyridines also include antitumour activity (Humphreys, S. R.; Vendeti, T. M.; Gotti, C. J.; Kline, J.; Goldin, A.; Kaplan, N. O., Canc. Res., 22, 1962, 483. Ross, W. C. J., J. Chem. Soc., 1965, 1816). 1,4-dihydropyridines have also been reported to possess analgesic and curare properties (Phillips, A. P., J. Amer. Chem. Soc., 71, 1949, 4003). This type of compounds also possesses CNS depressant (anticonvulsant and analgesic) activity (Swamay, S. K.; Reddy, T. M.; Reddy, V. M., Indian J. Pharm. Sci., 60, 1998, 102.). There are also reports of this class of compounds possessing antiasthmatic activity by reducing in vitro lipoperoxidation and in vivo experimental hyper-reactivity and cell infiltration (Cole, H. W.; Brown, C. E.; Magee, C.; Roudebush, R. E.; Bryant, H. U., Gen. Pharmacol., 26, 1995, 431.). Donkor et al. have reported the radioprotective effects of 1,4-dihydropyridines (Donkor, I. O.; Zhou, X; Schmidt, J.; Agarwal K. C.; Kishore, V., Bioorg. Med. Chem., 6, 1998, 563.). Albeit, even the simple 2,6-dimethyl-3,5-dicarboalkoxy-1,4-dihydropyridines have some hypotensive activity in anaesthetised animals, good activity is generally observed with compounds having a cyclic substituent in the 4-position, particularly 4-aryl compounds. Most active compounds were 4-heteroaryl and 4-ortho substituted phenyl derivatives. Activity usually decreases as the ortho substituent is moved to para position in the phenyl ring. Since compounds possessing both electron withdrawing and donating substituent in the ortho position of the 4-phenyl group are active, it is possible that the activity in these compounds may be enhanced by the presence of bulky groups which cause the 4-substituent to prefer an orientation perpendicular to the plane of the dihydropyridine ring. This theory is supported by the better activity shown by the 2,6-substituted compounds (25) and (26). Good oral activity was most constantly observed among the 4-heteroaryl and 4-ortho substituted phenyl compounds. The latter generally showed greater activity and fewer signs of toxicity.

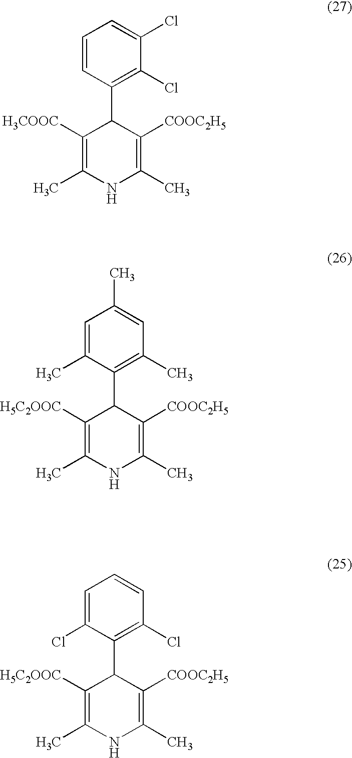

Dihydropyridines for example felodipine (27) show some structural features of certain diuretics. 1,4-dihydropyridines of nifedipine type are the most extensively investigated class of the calcium antagonists because of possibility of wide structural variations and superior potency. Second generation of dihydropyridine (DHP) development candidates having high potency are all chiral owing to non-identical ester functions (Meyer, H.; Bossert, F.; Wehinger, E.; Stoepel, K.; Vater, W.; Arzneim.-Forsch./Drug Res., 30, 1981, 407) and can exist in enantiomeric forms differing in absolute configuration at C-4 (Towart, R.; Wehinger, E.; Meyer, H.; Schmiedbergs, N., Arch. Pharmacol., 317, 1981, 183). Felodipine (27) is characterized by pronounced peripheral vasodilation, so it appears to be particularly interesting as antihypertensive (Ek, B.; Ahnoft, M.; Norlander, H. H.; Jung, B. L., Arch Pharmacol., 313, Suppl. R37, 1980).

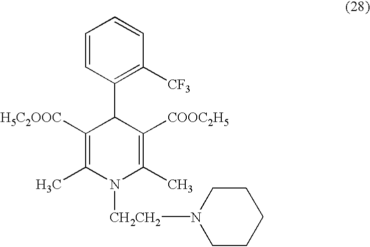

Floridipine (28) is the first DHP with a substituent on the nitrogen atom exhibits antihypertensive activity in rats and dogs when administered by oral route. An interesting correlation between the puckering of the DHP ring found by X-ray structure analysis and calcium antagonistic activity has also been studied in case of this compound. It has been reported that in the series of 4-phenyl substituted DHP's, an ortho substituent brings about a very slight deviation from the planarity of the DHP ring. This is an important criterion for higher activity (Fossheim, R, Sventag, K., Mastad A., romming C., Shefter E., Triggle D H, J. Med. Chem., 25, 1982, 126).

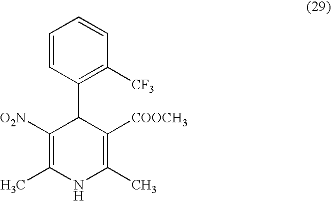

Structural modifications among dihydropyridines continue to concentrate on ester and aryl substituents with aim of discovering examples having slower onset and longer duration of action. It has been reported that some dihydropyridine analogues, instead of inhibiting, increase force of contraction. Bay K. 8644 (29) is the most thoroughly studied prototype of these agents (Preuss, K. C.; Gross, G. J.; Brooks, H. L.; Warltier, D. C., Life Sciences, 37,1985,1271. Schramm, M.; Thomas, G.; Towart, R.; Franchowiak G., Nature, 309, 1983, 535. Schramm, M.; Thomas, G.; Towart, R.; Franchowiak, G., Arzneim.-Forsch./Drug Res., 33, 1983, 1268. Preus, K. C.; Chang, N. L.; Brooks, H. L.; Warltier, D. C., J. Cardiovas. Pharmacol. 6,1985,531).

### OBJECTS OF THE INVENTION

The main object of the invention is to provide a process for the preparation of 1,4-dihydropyridines which is a single step reaction and obviates the drawbacks of the prior art.

It is another object of the invention to provide a process for the preparation of 1,4-dihydropyridines which is inexpensive, safe, environmentally friendly and provides a high yield.

It is another object of the invention to provide novel 1,4-dihydropyridines which are useful as therapeutic agents.

### SUMMARY OF THE INVENTION

Accordingly the present invention provides a process for the preparation of 1,4-dihydropyridines of the formula 1

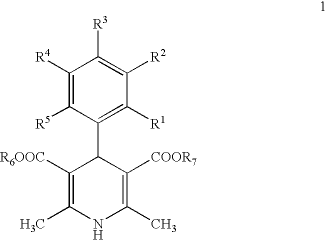

wherein R₁ is H, NO₂, Cl, OAc, OH, R₂ is H, NO₂, Cl, —O—CH₂—O—, OMe, OAc, OEt, OH, R₃ is H, NO₂, Cl, N(Me)₂, —O—CH₂—O—, OMe, OAc, OH, R₄ is H, OMe, OAc, OH, R₅ is H, Cl, I, and R₆ and R₇ are either methyl, ethyl or both, said process comprising, preparing a mixture of an aromatic aldehyde, allyl acetoacetate and a source of ammonia, adsorbing the prepared mixture on adsorbent till adsorbent becomes free flowing, heating the material obtained in step (i) under microwave irradiation at 250 to 600 W for 30 seconds to ten minutes, cooling the reaction mixture to room temperature and recovering the compound of formula I where in R₁ is H, NO₂, Cl, OAc, R₂ is H, NO₂, Cl, —O—CH₂—O—, OMe, OAc, OEt, R₃ is H, NO₂, Cl, N(Me)₂, —O—CH₂—O—, OMe, OAc, R₄ is OMe, Oac, R₅ is H Cl, I, and R₆ and R₇ are either methyl, ethyl or both, by hydrolysing the ester group in aromatic portion of compound using an hydrolysing agent to give corresponding hydroxy compounds of formula I where in R₁ is H, NO₂, Cl, OH, R₂ is H, NO₂, Cl, —O—CH₂—O—, OMe, OEt, OH, R₃ is H, NO₂, Cl, N(Me)₂, —O—CH₂—O—, OMe, OH, R₄ is H, OMe, OH, R₅ is Cl, I, and R₆ and R₇ are methyl, ethyl or both.

In one embodiment of the invention, the alkyl acetoacetate is selected from the group consisting of methyl acetoacetate, ethyl acetoacetate and a mixture thereof.

In another embodiment of the invention, step (iii) is carried out under microwave irradiation at 300 to 500 W to obtain compound of formula 1 wherein R₆ and R₇ are both methyl

In another embodiment of the invention, step (iii) is carried out under microwave irradiation at 350 to 600 W to obtain the compound of formula 1 wherein R₆ and R₇ are both ethyl.

In another embodiment of the invention, step (iii) is carried out under microwave irradiation at 250 to 400 W to obtain the compound of formula 1 wherein R₆ is methyl and R₇ is ethyl

In another embodiment of the present invention the aromatic aldehyde used is selected from the group consisting of benzaldehyde, 2-nitrobenzaldehyde, 3-nitrobenzaldehyde, 4-nitrobenzaldehyde, 2,3-dichlorobenzaldehyde, 2,4-dichlorobenzaldehyde, 2,6-dichlorobenzaldehyde, 4-(N,N-dimethyl)benzaldehyde, 3(4)-methylenedioxybenzaldehyde, 3,4,5-trimethoxy benzaldehyde, 2-nitro-3(4)-methylenedioxybenzaldehyde, 2-nitro-3(4)-trimethoxy benzaldehyde, 2-nitro-5-acetoxybenzaldehyde, 3-methoxy-4-acetobenzaldehyde, 3-acetoxy-4-methoxybenzaldehyde, 2-acetoxy-3-methoxybenzaldehyde, 4-acetoxy-5-iodo-3-methoxybenzaldehyde, 2-acetoxy-5-ethoxybenzaldehyde, 4-acetoxy-3-ethoxybenzaldehyde, 3-acetoxybenzaldehyde, 4-acetoxybenzaldehyde and 2,4-diacetoxyberzaldehyde.

In another embodiment of the invention the source of ammonia used is selected from the group consisting of ammonium acetate, ammonium acetate solution, ammonia anhydrous, ammonium hydroxide solution and any other source of ammonia.

In another embodiment of the invention adsorbent used is selected from the group consisting of basic alumina, neutral alumina and alkali metal carbonate.

In yet another embodiment of the invention the hydrolysing agent used is selected from ammonia and alkali hydroxide.

In still another embodiment of the invention, the reactant mixture is prepared by trituration, dissolving in solvent and removing solvent in vacuo, or by stirring with help of a stirrer.

In a further embodiment of the invention, compounds of formula 1 are recovered from reaction mixture by extracting with water immiscible organic solvent selected from the group consisting of chloroform, dichloromethane, ether and ethyl acetate.

The present invention also provides novel 1,4-dihydropyridines of the formula 1

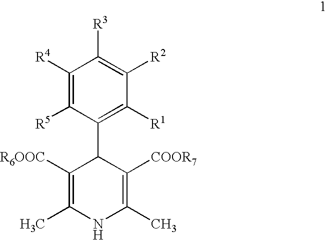

wherein R₁ is H, NO₂, Cl, OAc, OH, R₂ is H, NO₂, I, —O—CH₂—O—, OMe, OAc, OEt, OH, R₃ is H, NO₂, Cl, N(Me)₂, —O—CH₂—O—, OMe, OAc, OH, R₄ is H, OMe, OAc, OH, R₅ is H, Cl, I, and R₆ and R₇ are methyl ethyl or both.

In one embodiment of the invention, R₁, R₂, R₃, R₄, R₅ are given in the Table below

| R₁ | R₂ | R₃ | R₄ | R₅ | Alkyl acetoacetate R₆ & R₇ |
|---|---|---|---|---|---|
| H | —O—CH₂—O— | | H | H | CH₃ & CH₃, C₂H₅ & C₂H₅, CH₃ & C₂H₅ |
| NO₂ | H | —O—CH₂—O— | | H | CH₃ & CH₃, C₂H₅ & C₂H₅, CH₃ & C₂H₅ |
| NO₂ | OMe | OMe | OMe | H | CH₃ & CH₃, C₂H₅ & C₂H₅, CH₃ & C₂H₅ |
| NO₂ | H | H | OAc | H | CH₃ & CH₃, C₂H₅ & C₂H₅, CH₃ & C₂H₅ |
| H | OMe | OAc | H | H | CH₃ & CH₃, C₂H₅ & C₂H₅ |
| H | OAc | OMe | H | H | CH₃ & CH₃, C₂H₅ & C₂H₅ |
| Oac | OMe | H | H | H | CH₃ & CH₃, C₂H₅ & C₂H₅, CH₃ & C₂H₅ |
| H | OMe | OAc | H | I | CH₃ & CH₃, C₂H₅ & C₂H₅ |
| Oac | OEt | H | H | H | CH₃ & CH₃, C₂H₅ & C₂H₅, CH₃ & C₂H₅ |
| H | OEt | OAc | H | H | CH₃ & CH₃, C₂H₅ & C₂H₅, CH₃ & C₂H₅ |
| Oac | H | OAc | H | H | CH₃ & CH₃, C₂H₅ & C₂H₅, CH₃ & C₂H₅ |
| NO₂ | H | H | OH | H | CH₃ & CH₃, C₂H₅ & C₂H₅, CH₃ & C₂H₅ |
| H | OMe | OH | H | I | CH₃ & CH₃, C₂H₅ & C₂H₅, CH₃ & C₂H₅ |
| OH | OEt | H | H | H | CH₃ & CH₃, C₂H₅ & C₂H₅, CH₃ & C₂H₅ |
| H | OEt | OH | H | H | CH₃ & CH₃, C₂H₅ & C₂H₅, CH₃ & C₂H₅ |
| OH | H | OH | H | H | CH₃ & CH₃, C₂H₅ & C₂H₅, CH₃ & C₂H₅ |
| H | OH | H | H | H | CH₃ & C₂H₅ |
| H | H | OH | H | H | CH₃ & C₂H₅ |
| OH | OMe | H | H | H | CH₃ & C₂H₅ |
| H | OAc | H | H | H | CH₃ & C₂H₅ |
| H | H | OAc | H | H | CH₃ & C₂H₅ |

### DETAILED DESCRIPTION OF THE INVENTION

The aromatic aldehydes used for preparation of dihydropyridines according to the invention are of the formula given below and exemplified in Table 1

TABLE 1

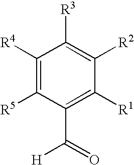

| | R¹ | R² | R³ | R⁴ | R⁵ |
|---|---|---|---|---|---|
| 1A | H | H | H | H | H |
| 2A | NO₂ | H | H | H | H |
| 3A | H | NO₂ | H | H | H |
| 4A | H | H | NO₂ | H | H |
| 5A | Cl | Cl | H | H | H |
| 6A | Cl | H | Cl | H | H |
| 7A | CI | H | H | H | Cl |
| 8A | H | H | N(Me)₂ | H | H |
| 9A | H | —O—CH₂—O— | | H | H |
| 10A | H | OMe | OMe | OMe | H |
| 11A | NO₂ | H | —O—CH₂—O— | | H |
| 12A | NO₂ | OMe | OMe | OMe | H |
| 13A | NO₂ | H | H | OAc | H |
| 14A | H | OMe | OAc | H | H |
| 15A | H | OAc | OMe | H | H |
| 16A | OAc | OMe | H | H | H |
| 17A | H | OMe | OAc | H | I |
| 18A | OAc | OEt | H | H | H |
| 19A | H | OEt | OAc | H | H |
| 20A | H | OAc | H | H | H |
| 21A | H | H | OAc | H | H |
| 22A | OAc | H | OAc | H | H |

The source of ammonia used can be ammonium acetate, ammonium acetate solution, ammonia anhydrous, ammonium hydroxide solution and any other source of ammonia. Adsorbent used may be such as basic alumina, neutral alumina, alkali metal carbonate, or any other basic adsorbent. Hydrolysing agent used may be such as ammonia, alkali hydroxide. The method of preparation of the mixture of the reactants may be such as trituration, dissolving in solvent and removing the solvent in vacuo, stirring with help of stirrer. Compounds of formula I may be recovered from reaction mixture by extracting with water immiscible organic solvent such as chloroform, dichloromethane, ether, ethyl acetate. Reaction scheme 1 below illustrates the process of invention. Compounds prepared by process of the invention are given in Tables 2, 3 and 4 below where alkyl acetoacetate is methylacetoacetate, ethylacetoacetate or a mixture thereof respectively.

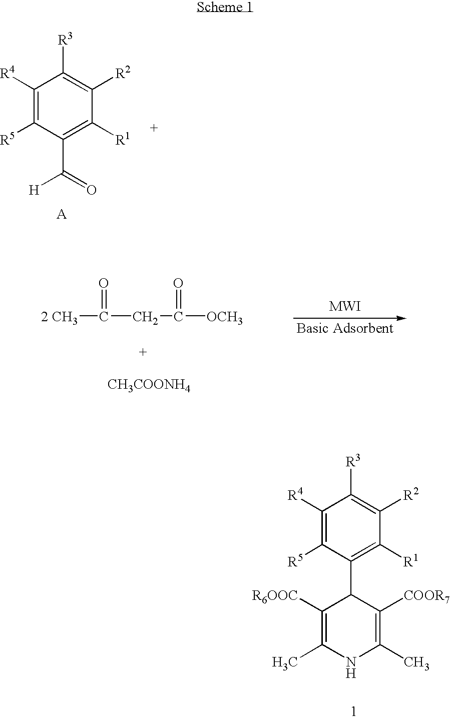

TABLE 2
when R6 and R7 are both methyl acetoacetate (*New compounds)

| | R¹ | R² | R³ | R⁴ | R⁵ | m.p. (° C.) | Molecular Formula |
|---|---|---|---|---|---|---|---|
| 1B | H | H | H | H | H | 193 | C₁₇H₁₉NO₄ |
| 2B | NO₂ | H | H | H | H | 174 | C₁₇H₁₈N₂O₆ |
| 3B | H | NO₂ | H | H | H | 203 | C₁₇H₁₈N₂O₆ |
| 4B | H | H | NO₂ | H | H | 195 | C₁₇H₁₈N₂O₆ |
| 5B | Cl | Cl | H | H | H | 186 | C₁₇H₁₇NO₄ Cl₂ |
| 6B | Cl | H | Cl | H | H | 183 | C₁₇H₁₇NO₄ Cl₂ |
| 7B | Cl | H | H | H | Cl | 193 | C₁₇H₁₇NO₄ Cl₂ |
| 8B | H | H | N(Me)₂ | H | H | 186 | C₁₉H₂₄N₂O₄ |
| 9B* | H | —O—CH₂—O— | | H | H | 182 | C₁₈H₁₉N₂O₆ |
| 10B | H | OMe | OMe | OMe | H | 136 | C₂₀H₂₅NO₇ |
| 11B* | NO₂ | H | —O—CH₂—O— | | H | 210 | C₁₈H₁₈N₂O₈ |
| 12B* | NO₂ | OMe | OMe | OMe | H | 170 | C₂₀H₂₄N₂O₉ |
| 13B* | NO₂ | H | H | OAc | H | 200 | C₁₉H₂₀N₂O₈ |
| 14B* | H | OMe | OAc | H | H | 215 | C₂₀H₂₃NO₇ |
| 15B* | H | OAc | OMe | H | H | 170 | C₂₀H₂₃NO₇ |
| 16B* | OAc | OMe | H | H | H | 162 | C₂₀H₂₃NO₇ |
| 17B* | H | OMe | OAc | H | I | 230 | C₂₀H₂₂NO₇I |
| 18B* | OAc | OEt | H | H | H | 173 | C₂₁H₂₅NO₇ |
| 19B* | H | OEt | OAc | H | H | 197 | C₂₁H₂₅NO₇ |
| 20B | H | OAc | H | H | H | 182 | C₁₉H₂₁NO₆ |
| 21B | H | H | OAc | H | H | 195 | C₁₉H₂₁NO₆ |
| 22B* | OAc | H | OAc | H | H | 249 | C₂₁H₂₃NO₈ |
| 23B* | NO₂ | H | H | OH | H | 207 | C₁₇H₁₈N₂O₇ |
| 24B | H | OMe | OH | H | H | 213 | C₁₈H₂₁NO₆ |
| 25B | H | OH | OMe | H | H | 173 | C₁₈H₂₁NO₆ |
| 26B | OH | OMe | H | H | H | 170 | C₁₈H₂₁NO₆ |
| 27B* | H | OMe | OH | H | I | 235 | C₁₈H₂₀NO₆I |
| 28B* | OH | OEt | H | H | H | 179 | C₁₉H₂₃NO₆ |
| 29B* | H | OEt | OH | H | H | 203 | C₁₉H₂₃NO₆ |
| 30B | H | OH | H | H | H | 191 | C₁₇H₁₉NO₅ |
| 31B | H | H | OH | H | H | 205 | C₁₇H₁₉NO₅ |
| 32B* | OH | H | OH | H | H | 262 | C₁₇H₁₉NO₆ |

TABLE 3
when R6 is methyl acetoacetate and R7 is ethyl acetoacetate (*New compounds)

| | R¹ | R² | R³ | R⁴ | R⁵ | m.p. (° C.) | Molecular formula |
|---|---|---|---|---|---|---|---|
| 1B | H | H | H | H | H | 147 | C₁₈H₂₁NO₄ |
| 2B | NO₂ | H | H | H | H | 136 | C₁₈H₂₀N₂O₆ |
| 3B | H | NO₂ | H | H | H | 174 | C₁₈H₂₀N₂O₆ |
| 4B | H | H | NO₂ | H | H | 159 | C₁₈H₂₀N₂O₆ |
| 5B | Cl | Cl | H | H | H | 145 | C₁₈H₁₉NO₄ Cl₂ |
| 6B | Cl | H | Cl | H | H | 130 | C₁₈H₁₉NO₄ Cl₂ |
| 7B | Cl | H | H | H | Cl | 170 | C₁₈H₁₉NO₄ Cl₂ |
| 8B | H | H | N(Me)₂ | H | H | 142 | C₂₀H₂₆N₂O₄ |
| 9B* | H | —O—CH₂—O— | | H | H | 157 | C₁₉H₂₁N₂O₆ |
| 10B* | H | OMe | OMe | OMe | H | 102 | C₂₁H₂₇NO₇ |
| 11B* | NO₂ | H | —O—CH₂—O— | | H | 182 | C₁₉H₂₀N₂O₈ |
| 12B* | NO₂ | OMe | OMe | OMe | H | 130 | C₂₁H₂₆N₂O₉ |
| 13B* | NO₂ | H | H | OAc | H | 163 | C₂₀H₂₂N₂O₈ |
| 14B | H | OMe | OAc | H | H | 188 | C₂₁H₂₅NO₇ |
| 15B | H | OAc | OMe | H | H | 133 | C₂₁H₂₅NO₇ |
| 16B* | OAc | OMe | H | H | H | 123 | C₂₁H₂₅NO₇ |
| 17B | H | OMe | OAc | H | I | 201 | C₂₁H₂₄NO₇I |
| 18B* | OAc | OEt | H | H | H | 138 | C₂₂H₂₇NO₇ |
| 19B* | H | OEt | OAc | H | H | 159 | C₂₂H₂₇NO₇ |
| 20B* | H | OAc | H | H | H | 150 | C₂₀H₂₃NO₆ |
| 21B* | H | H | OAc | H | H | 162 | C₂₀H₂₃NO₆ |
| 22B* | OAc | H | OAc | H | H | 209 | C₂₂H₂₅NO₈ |
| 23B* | NO₂ | H | H | OH | H | 171 | C₁₈H₂₀N₂O₇ |
| 24B | H | OMe | OH | H | H | 178 | C₁₉H₂₃NO₆ |
| 25B | H | OH | OMe | H | H | 142 | C₁₉H₂₃NO₆ |
| 26B* | OH | OMe | H | H | H | 125 | C₁₉H₂₃NO₆ |
| 27B* | H | OMe | OH | H | I | 196 | C₁₉H₂₂NO₆I |
| 28B* | OH | OEt | H | H | H | 143 | C₂₀H₂₅NO₆ |
| 29B* | H | OEt | OH | H | H | 160 | C₂₀H₂₅NO₆ |
| 30B* | H | OH | H | H | H | 145 | C₁₈H₂₁NO₅ |
| 31B* | H | H | OH | H | H | 168 | C₁₈H₂₁NO₅ |
| 32B* | OH | H | OH | H | H | 220 | C₁₈H₂₁NO₆ |

TABLE 4
when R6 and R7 are both ethyl acetoacetate (*New compounds)

| | R¹ | R² | R³ | R⁴ | R⁵ | m.p. (° C.) | Molecular formula |
|---|---|---|---|---|---|---|---|
| 1B | H | H | H | H | H | 155 | C₁₉H₂₃NO₄ |
| 2B | NO₂ | H | H | H | H | 140 | C₁₉H₂₂N₂O₆ |
| 3B | H | NO₂ | H | H | H | 168 | C₁₉H₂₂N₂O₆ |
| 4B | H | H | NO₂ | H | H | 152 | C₁₉H₂₂N₂O₆ |
| 5B | Cl | Cl | H | H | H | 148 | C₁₉H₂₁NO₄ Cl₂ |
| 6B | Cl | H | Cl | H | H | 140 | C₁₉H₂₁NO₄ Cl₂ |
| 7B | Cl | H | H | H | Cl | 153 | C₁₉H₂₁NO₄ Cl₂ |
| 8B | H | H | N(Me)₂ | H | H | 151 | C₂₁H₂₈N₂O₄ |
| 9B* | H | —O—CH₂—O— | | H | H | 137 | C₂₀H₂₃N₂O₆ |
| 10B | H | OMe | OMe | OMe | H | 105 | C₂₂H₂₉NO₇ |
| 11B* | NO₂ | H | —O—CH₂—O— | | H | 177 | C₂₀H₂₂N₂O₈ |
| 12B* | NO₂ | OMe | Ome | OMe | H | 133 | C₂₂H₂₈N₂O₉ |
| 13B* | NO₂ | H | H | OAc | H | 172 | C₂₁H₂₄N₂O₈ |
| 14B* | H | OMe | OAc | H | H | 180 | C₂₂H₂₇NO₇ |
| 15B* | H | OAc | OMe | H | H | 133 | C₂₂H₂₇NO₇ |
| 16B* | OAc | OMe | H | H | H | 135 | C₂₂H₂₇NO₇ |
| 17B* | H | OMe | OAc | H | I | 190 | C₂₂H₂₆NO₇I |
| 18B* | OAc | OEt | H | H | H | 146 | C₂₃H₂₉NO₇ |
| 19B* | H | OEt | OAc | H | H | 157 | C₂₃H₂₉NO₇ |
| 20B | H | OAc | H | H | H | 140 | C₂₁H₂₅NO₆ |
| 21B | H | H | OAc | H | H | 155 | C₂₁H₂₅NO₆ |
| 22B* | OAc | H | OAc | H | H | 207 | C₂₃H₂₇NO₈ |
| 23B* | NO₂ | H | H | OH | H | 175 | C₁₉H₂₂N₂O₇ |
| 24B | H | OMe | OH | H | H | 188 | C₂₀H₂₅NO₆ |
| 25B | H | OH | OMe | H | H | 139 | C₂₀H₂₅NO₆ |
| 26B | OH | OMe | H | H | H | 140 | C₂₀H₂₅NO₆ |
| 27B* | H | OMe | OH | H | I | 199 | C₂₀H₂₄NO₆I |
| 28B* | OH | OEt | H | H | H | 147 | C₂₁H₂₇NO₆ |
| 29B* | H | OEt | OH | H | H | 160 | C₂₁H₂₇NO₆ |
| 30B | H | OH | H | H | H | 149 | C₁₉H₂₃NO₅ |
| 31B | H | H | OH | H | H | 160 | C₁₉H₂₃NO₅ |
| 32B* | OH | H | OH | H | H | 212 | C₁₉H₂₃NO₆ |

The process for the preparation of 1,4-dihydropyridines of the formula I where in R₁ is H, NO₂, Cl, OAc, OH, R₂ is H, NO₂, Cl, —O—CH₂—O—, OMe, OAc, OEt, OH, R₃ is H, NO₂, Cl, N(Me)₂, —O—CH₂—O—, OMe, OAc, OH, R is H, OMe, OAc, OH and R₅ is H, Cl, I, is carried out under dry conditions on a solid support under the influence of the microwave irradiations. No solvent is used as a medium for reaction. The process is generally carried out by taking one mole of aldehyde (1A to 22A in Table 1) in a mortar and adding 2.2 moles of methylacetoacetate or ethylacetoacetate or a mixture thereof to it. The two are then mixed thoroughly with the help of a pestle in a mortar. Ammonium acetate (1.2 moles) is then added to the above reaction mixture and the mixture then triturated with the help of a pestle. A basic adsorbent such as potassium carbonate, calcium carbonate, aluminium oxide or magnesium oxide is then added in small increments with thorough mixing so as to adsorb whole of the above mixture on it till the adsorbent becomes free flowing. The adsorbent is then transferred into a conical flask much larger in capacity as compared to the volume of the adsorbent. A funnel was placed on the flask as condenser. The flask was then placed in a microwave oven cavity. Another flask containing ice (as heat sink) with a funnel as condenser was also placed in the microwave cavity along with the reaction flask (Heat sink is to be placed only if the quantity of the reactants is less. If sufficient quantity of reactants is there to adsorb all the microwaves then heat sink is not required). The reaction vessel was then subjected to microwave irradiations (MWI) at 250 to 600 W for six minutes depending on the reactants and the reaction vessel was then allowed to cool to room temperature. The compound obtained was then extracted with a water-immiscible organic solvent after shaking it thoroughly with adsorbent or stirring it on a magnetic stirrer. The organic solvent extract was filtered through a Buchner funnel on a filter paper and the organic solvent layer washed with adequate quantity of water. Organic solvent layer was then dried over anhydrous sodium sulphate or anhydrous magnesium sulphate and the extract filtered and the solvent removed by distillation under vacuum to give residue. The residue obtained above is then taken in aprotic polar solvent to give light yellow crystals of product. Acetate group m compounds can be subjected to hydrolysis by stirring one mole of the compound with 1.1 mole of ammonium hydoxide solution in aprotic polar solvent for 15 min to 75 min at 30-35° C. on a magnetic stirrer and then removing the solvent under vacuum. The compounds were recrystallised in petroleum ether to give compounds respectively. All steps for processing of product are done in dark chamber or in red light to avoid decomposition of the compound by daylight/U.V. rays to achieve high yields. Reaction is carried out in glassware, earthenware, ceramic or plastic containers marked as microwave safe shaped so as to prevent escape of reactants or products in vapour form during reaction by effectively controlling the power output. The process of preparation of 1,4-dihydropyridines is described in detail below by way of illustrative examples and should not be construed to limit the scope of the present invention.

### EXAMPLE 1

One mmole of 2-nitrobenzaldehyde was taken in a mortar and to it 2.2 mmoles of methyl acetoacetate was added and mixed thoroughly with help of pestle in a mortar. Ammonium acetate (1.2 mmoles) was added to reaction mixture and this mixture was then triturated with help of pestle. To this mixture aluminum oxide was added in small increments with thorough mixing till mixture became free flowing mixture was then transferred to a conical flask much larger in capacity compared to volume of adsorbent A glass funnel was placed on the flask as condenser. Reaction mixture was subjected to microwave irradiation at 400W for 6 min. a microwave oven placing a heat sink along with it. Reaction mixture was then allowed to cool to room temperature and the compound extracted with 3×50 ml portions of dichloromethane after stirring it thoroughly with adsorbent. The organic solvent extract was filtered through a filter paper on Buchner funnel and the organic solvent layer washed with 2×100 ml portions of water and the organic layer then dried over anhydrous sodium sulphate. Extract was filtered and solvent removed by distillation under vacuum to give residue which was recrystallised in methanol to, give yellow coloured crystals of 4-(2-nitro)phenyl-2,6-dimethyl-3,5-dicarbomethoxy-1,4-dihydropyridine, yield 90%. m.p. 178° C.

### EXAMPLE 2

1 mmole of 2-acetoxy-3-methoxybenzaldehyde was taken in mortar and to it 2.2 mmoles of methyl acetoacetate was added and mixed thoroughly with help of pestle in mortar. Ammonium acetate (1.2 mmoles) was added to reaction mixture and this mixture then triturated with help of pestle. Calcium carbonate was added in small increments with thorough mixing till mixture became free flowing. Mixture was transferred to a conical flask much larger in capacity compared to volume of adsorbent. A glass funnel was placed on the flask as condenser. Reaction mixture was subjected to microwave irradiation at 340W for 7 min in microwave oven placing a heat sink along with it. Reaction mixture was allowed to cool to room temperature and the compound extracted with 3×50 ml portions of chloroform after stirring it thoroughly with adsorbent. The organic solvent extract was filtered through filter paper on Buchner funnel and the chloroform layer washed with 2×100 ml portions of water. The organic layer was dried over anhydrous sodium sulphate and the extract filtered and solvent removed by distillation under vacuum to give residue which was recrystallised in methanol to give yellow coloured crystals of 4-(2-acetoxy-3-methoxy)phenyl-2,6-dimethyl-3,5-dicarbomethoxy-1,4-dihydropyridine in 85% yield. m.p. 162° C.

### EXAMPLE 3

1 mmole of 2-acetoxy-3-methoxybenzaldehyde was taken in a mortar and to it 2.2 mmoles of methyl acetoacetate was added and mixed thoroughly with help of pestle in mortar. Ammonium acetate (1.2 mmoles) was added to above reaction mixture and this mixture then triturated with help of pestle. Calcium carbonate was then added in small increments with thorough mixing till mixture became free flowing. Mixture was transferred to a conical flask much larger in capacity compared to volume of adsorbent. A glass funnel was placed on the flask as condenser. Reaction mixture was subjected to microwave irradiation at 340W for 7 min. in a microwave oven placing a heat sink along with it. Allowed the reaction mixture to cool to the room temperature. Extracted the compound with 3×50 ml portions of chloroform after stirring it thoroughly with adsorbent. Filtered the organic solvent extract through a filter paper on Buchner funnel. Washed the organic solvent layer with 2×100 ml portions of water. Dried organic solvent layer over anhydrous sodium sulphate. Filtered the extract and removed solvent by distillation under vacuum to give residue which was recrystallised in methanol to give yellow coloured crystals of 4-(2-acetoxy-3-methoxy)phenyl-2,6-dimethyl-3,5-dicarbomethoxy-1,4-dihydropyridine in 85% yield. m.p. 162° C. 4-(2-acetoxy-3-methoxy)phenyl-2,6-dimethyl-3,5-dicarbomethoxy-1,4-dihydropyridine was subjected to hydrolysis by stirring one mole of compound with 1.1 mole of ammonium hydroxide solution in methanol for one hour at 30-35° C. on a magnetic stirrer and then removing solvent under vacuum. Resulting compound was recrystallised in petroleum ether to give 4-(2-hydroxy-3-methoxy)phenyl-2,6-dimethyl-3,5-dicarbomethoxy-1,4-dihydropyridine m.p. 170° C. yield 80%.

### EXAMPLE 4

1 mmole of benzaldehyde was taken in mortar and to it 2.2 mmoles of methyl acetoacetate was added and mixed thoroughly with help of pestle in mortar. Ammonium hydroxide (1.2 mmoles) was added to reaction mixture and this mixture then triturated with help of pestle. Aluminum oxide was then added in small increments with thorough mixing till mixture became free flowing. Mixture was transferred to a conical flask much larger in capacity compared to volume of adsorbent. A glass funnel was placed on the flask as condenser. Reaction mixture was subjected to microwave irradiation at 340W for 4 min 30 sec in a microwave oven placing a heat sink along with it. Allowed the reaction mixture to cool to the room temperature. Extracted the compound with 3×50 ml portions of ethyl acetate after stirring it thoroughly with adsorbent. Filtered the organic solvent extract through a filter paper on the Buchner funnel. Washed the organic layer with 2×100 ml portions of water. Dried the organic solvent layer over anhydrous sodium sulphate. Filtered the extract and removed the solvent by distillation under vacuum to give the residue which was recrystallised in methanol to give yellow coloured crystals of 4-phenyl-2,6-dimethyl-3,5-dicarbomethoxy-1,4-dihydropyridine yield 87%. m.p. 193° C.

### EXAMPLE 5

Same procedure as Example 1 was followed except that instead of methyl acetoacetate, premixed mixture of methyl acetoacetate and ethyl acetoacetate (both 1.1 mmoles) was used. Reaction mixture was subjected to same procedure as Example 1 to give yellow coloured crystals of 4-(2-nitrophenyl)-2,6-dimethyl-3-carboethoxy-5-carbomethoxy-1,4,-dihydropyridines. m.p. 178° C. in 90% yield.

### EXAMPLE 6

Same procedure as Example 2 above was followed except that instead of methyl acetoacetate, premixed mixture of methyl acetoacetate and ethyl acetoacetate (both 1.1 mmoles) was used. Also, instead of calcium carbonate aluminum oxide was added in small increments with thorough mixing till mixture became free flowing. Microwave irradiation was done at 300W for 9 min in microwave oven placing a heat sink along with it. Compound from cooled reaction mixture was extracted with with 3×50 ml portions of dichloromethane instead of chloroform. The residue on recrystallisation in methanol yielded yellow coloured crystals of 4-(2-actoxy-3-methoxyphenyl)-2,6-dimethyl-3 carboethoxy-5-carbomethoxy-1,4-dihydropyridines (m.p. 162° C.) in 85% yield.

### EXAMPLE 7

Same procedure as Example 3 was followed except that instead of methyl acetoacetate, premixed mixture of methyl acetoacetate and ethyl acetoacetate (both 1.1 mmoles) was used. Also, instead of calcium carbonate, aluminum oxide was added in small increments with thorough mixing till mixture became free flowing. Microwave irradiation was done at 300W for 9 min in microwave oven placing a heat sink along with it. Compound from cooled reaction mixture was extracted with 3×50 ml portions of dichloromethane and not chloroform. Residue on recrystallisation in methanol yielded yellow coloured crystals of 4-(2-actoxy-3-methoxyphenyl)2,6-dimethyl-3-carboethoxy-5-carbomethoxy-1,4,-dihydropyridines (m.p.162° C.) in 85% yield. Acetoxy compound was subjected to hydrolysis by stirring 1 mole of compound with 1.1 mole of ammonium hydroxide solution in methanol for 1 hr at 30-35° C. on a magnetic stirrer and then removing solvent under vacuum. Resulting compound was recrystallised in petroleum ether to give 4-2-hydroxy-3-methoxyphenyl)-2,6-dimethyl-3-carboethoxy-5-carbomethoxy-1,4-dihydropyridines. m. p. 170° C. in 75% yield.

### EXAMPLE 8

Same procedure as Example 1 was followed except that instead of methyl acetoacetate, ethyl acetoacetate (2.2 mmoles) was used. Reaction mixture was subjected to same procedure as in Example 1 except that microwave radiation was done at 460 W for 6 min and compound extracted with chloroform instead of dichloroethane. The product obtained was yellow coloured crystals of 4-(2-nitrophenyl)-2,6-dimethyl-3,5-dicarboethoxy-1,4,-dihydropyridines (m.p. 140° C.) in 85% yield.

### EXAMPLE 9

Same procedure as Example 2 was followed except that instead of methyl acetoacetate, ethyl acetoacetate (2.2 mmoles) was used. Microwave irradiation was done at 400W for 7 min. Compound from cooled reaction mixture was extracted with 3×50 ml portions of dichloromethane instead of chloroform. Residue on recrystallisation in methanol yielded yellow coloured crystals of 4-(2-acetoxy-3-methoxyphenyl)-2,6-dimethyl-3,5-dicarboethoxy-1,4, dihydropyridines. m.p. 135° C. in 80% yield.

### EXAMPLE 10

Same procedure as in Example 3 was followed except that instead of methyl acetoacetate, ethyl acetoacetate (2.2 mmoles) was used. Microwave irradiation was done at 400W for seven minutes. The compound from the cooled reaction mixture was extracted with with 3×50 ml portions of dichloromethane instead of chloroform. The residue on recystallisation in methanol yielded yellow coloured crystals of 4-(2-acetoxy-3-methoxyphenyl)-2,6-dimethyl-3,5-dicarboethoxy-1,4-dihydropyridines (m.p. 135° C.) in 80% yield. The acetoxy compound was subjected to hydrolysis by stirring one mole of compound with 1.1 mole of ammonium hydroxide solution in methanol for one hour at 30-35° C. on a magnetic stirrer and then removing the solvent under vacuum. The resulting compound was recrystallised in petroleum ether to give 4-2-hydroxy-3-methoxyphenyl)-2,6-dimethyl-3,5-carboethoxy-1,4,-dihydropyridines (m. p. 140° C.) in 75% yield.

The Main Advantages of the Present Invention are:

1. The reaction time is less than reported art (from 16 hrs to less than 10 minutes).

2. The yields of DHP compounds are high (80-90%) as compared to 70-80% in prior art.

3. Use of solvents is minimized as these are required only for extraction and crystallisation.

4. Hazard of fire is avoided since no solvents are used in the reaction

5. Some of the compounds obtained are novel and useful as therapeutic agents particularly antianginal and hypotensive agents or potentially useful as new test models to develop agents which could be used as drugs for the treatment of various cardiovascular ailments.

6. The entire process of synthesis is environment friendly.

7. The compounds on preliminary screening show excellent cardiovascular activity, the hydroxy compounds being particularly promising.

We claim:

1. A process for the preparation of 1,4-dihydropyridines of the formula 1 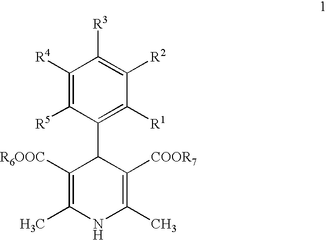
wherein R₁ is H, NO₂, Cl, OAc, OH, R₂ is H, NO₂, CL, —O—CH₂—O—, OMe, OAc, OEt, OH, R₃ is H, NO₂, Cl, N(Me)₂, —O—CH₂—O—, OMe, OAc, OH, R₄ is H, OMe, OAc, OH, R₅ is H, CL, I, and R₆ and R₇ are either methyl, ethyl or both, said process comprising
(i) preparing a mixture of an aromatic aldehyde, alkyl acetoacetate and a source of ammonia,
(ii) adsorbing the above prepared mixture on adsorbent till adsorbent becomes free flowing,
(iii) heating the material obtaining in step (ii) under microwave irradiations at 250 to 600 W for 30 seconds to ten minutes
(iv) cooling the above reaction mixture to room temperature and recovering the compound of formula I where in R₁ is H, NO₂, Cl, OAc, R₂ is H, NO₂, Cl, —O—CH₂—O—, OMe, OAc, OEt, R₃ is H, NO₂, CL, N(Me)₂, —O—CH₂—O—, OMe, OAc, R₄ is H, OMe, Oac, R₅ is H, Cl, I, and R₆ and R₇ are either methyl, ethyl or both, by hydrolysing the ester group in aromatic portion of compound using an hydrolysing agent to give corresponding hydroxy compounds of formula I wherein R₁ is H, NO₂, Cl, OH, R₂ is H, NO₂, Cl, —O—CH₂—O—, OMe, OEt, OH, R₃ is H, NO₂, Cl, N(Me)₂, —O—CH₂—O—, OMe, OH, R₄ is H, OMe, OH, R₅ is H Cl, I, and R₆ and R₇ are either methyl ethyl or both.

2. A process as claimed in claim 1 wherein the alkyl acetoacetate is selected from the group consisting of methyl acetoacetate, ethyl acetoacetate and a mixture thereof.

3. A process as claimed in claim 1 wherein step (iii) is carried out under microwave irradiation at 300 to 500 W to obtain the compound of formula 1 wherein R₆ and R₇ are both methyl.

4. A process as claimed in claim 1 wherein step (iii) is carried out under microwave irradiation at 350 to 600 W to obtain the compound of formula 1 wherein R₆ and R₇ are both ethyl.

5. A process as claimed in claim 1 wherein step (iii) is carried out under microwave irradiation at 250 to 400 W to obtain the compound of formula 1 wherein R₆ methyl and R₇ is ethyl.

6. A process as claimed in claim 1 wherein the aromatic aldehyde used is selected from the group consisting of benzaldehyde, 2-nitrobenzaldehyde, 3-nitrobenzaldehyde, 4-nitrobenzaldehyde, 2,3-dichlorobenzaldehyde, 2,4-dichlorobenzaldehyde, 2,6-dichlorobenzaldehyde, 4-N,N-dimethyl)benzaldehyde, 3(4)-methylenedioxybenzaldehyde, 3,4,5-trimethoxy benzaldehyde, 2-nitro-3(4)-methylenedioxybenzaldehyde, 2-nitro-3(4)-trimethoxy benzaldehyde, 2-nitro-5-acetoxybenzaldehyde, 3-methoxy-4-acetoxybenzaldehyde, 3-acetoxy-4-methoxybenzaldehyde, 2-acetoxy-3-methoxybenzaldehyde, 4-acetoxy-5-iodo-3-methoxybenzaldehyde, 2-acetoxy-5-ethoxybenzaldehyde, 4-acetoxy-3-ethoxybenzaldehyde, 3-acetoxybenzaldehyde, 4-acetoxybenzaldehyde and 2,4-diacetoxybenzaldehyde.

7. A process as claimed in claim 1 wherein the source of ammonia used is selected from the group consisting of ammonium acetate, ammonium acetate solution, ammonia anhydrous, ammonium hydroxide solution and any other source of ammonia.

8. A process as claimed in claim 1 wherein the adsorbent used is selected from the group consisting of basic alumina, neutral alumina and alkali metal carbonate.

9. A process as claimed in claim 1 wherein the hydrolysing agent used is selected from ammonia and alkali hydroxide.

10. A process as claimed in claim 1 wherein the reactant mixture is prepared by trituration, dissolving in solvent and removing solvent in vacuo, or by stirring with help of a stirrer.

11. A process as claimed in claim 1 wherein the compounds of formula I are recovered from the reaction mixture by extracting with water immiscible organic solvent selected from the group consisting of chloroform, dichloromethane, ether and ethyl acetate.

12. A process as claimed in claim 1 wherein the compound of formula 1 is recovered in step (iv) by extraction of residue using water immiscible solvent such as haloalkanes, ethers, ketones, alkylacetoacetates.

13. A process as claimed in claim 1 wherein when R₁, R₂, R₃, R₄, R₅, are as given in the table below, the acetyl group of the resultant compounds is hydrolysed in the presence of a base selected from ammonium hydroxide and alkali metal hydroxide at 20-40° C. in a polar protic solvent selected from an alkanol with having one to three carbon atoms.

| | | | | |
|---|---|---|---|---|
| NO₂ | H | H | OAc | H |
| H | OMe | OAc | H | H |
| H | OAc | OMe | H | H |
| OAc | OMe | H | H | H |
| H | OMe | OAc | H | I |
| OAc | OEt | H | H | H |
| H | OEt | OAc | H | H |
| H | OAc | H | H | H |
| H | H | OAc | H | H |
| OAc | H | OAc | H | H |

14. Novel 1,4-dihydropyridines of the formula 1 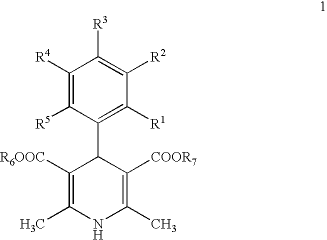
wherein R₁ is H, NO₂, Cl, OAc, OH, R₂ is H, NO₂, Cl, —O—CH₂—O—, OMe, OAc, OEt, OH, R₃ is H, NO₂, Cl, N(Me)₂, —O—CH₂—O—, OMe, OAc, OH, R₄ is H OMe, OAc, OH, R₅ is H, Cl, I, and R₆ and R₇ are either methyl, ethyl or both.

15. Novel 1,4-dihydropyridines as claimed in claim 14 wherein R₁, R₂, R₃, R₄, R₅, R₆ & R₇ are as given in the Table below

| R₁ | R₂ | R₃ | R₄ | R₅ | Alkyl acetoacetate (R₆ & R₇) |
|---|---|---|---|---|---|
| H | —O—CH₂—O— | | H | H | CH₃ & CH₃, C₂H₅ & C₂H₅, CH₃ & C₂H₅ |
| NO₂ | H | —O—CH₂—O— | | H | CH₃ & CH₃, C₂H₅ & C₂H₅, CH₃ & C₂H₅ |
| NO₂ | OMe | OMe | OMe | H | CH₃ & CH₃, C₂H₅ & C₂H₅, CH₃ & C₂H₅ |
| NO₂ | H | H | OAc | H | CH₃ & CH₃, C₂H₅ & C₂H₅, CH₃ & C₂H₅ |
| H | OMe | OAc | H | H | CH₃ & CH₃, C₂H₅ & C₂H₅ |
| H | OAc | OMe | H | H | CH₃ & CH₃, C₂H₅ & C₂H₅ |
| Oac | OMe | H | H | H | CH₃ & CH₃, C₂H₅ & C₂H₅, CH₃ & C₂H₅ |
| H | OMe | OAc | H | I | CH₃ & CH₃, C₂H₅ & C₂H₅ |
| Oac | OEt | H | H | H | CH₃ & CH₃, C₂H₅ & C₂H₅, CH₃ & C₂H₅ |
| H | OEt | OAc | H | H | CH₃ & CH₃, C₂H₅ & C₂H₅, CH₃ & C₂H₅ |
| Oac | H | OAc | H | H | CH₃ & CH₃, C₂H₅ & C₂H₅, CH₃ & C₂H₅ |
| NO₂ | H | H | OH | H | CH₃ & CH₃, C₂H₅ & C₂H₅, CH₃ & C₂H₅ |
| H | OMe | OH | H | I | CH₃ & CH₃, C₂H₅ & C₂H₅, CH₃ & C₂H₅ |
| OH | OEt | H | H | H | CH₃ & CH₃, C₂H₅ & C₂H₅, CH₃ & C₂H₅ |
| H | OEt | OH | H | H | CH₃ & CH₃, C₂H₅ & C₂H₅, CH₃ & C₂H₅ |
| OH | H | OH | H | H | CH₃ & CH₃, C₂H₅ & C₂H₅, CH₃ & C₂H₅ |
| H | OH | H | H | H | CH₃ & C₂H₅ |
| H | H | OH | H | H | CH₃ & C₂H₅ |
| OH | OMe | H | H | H | CH₃ & C₂H₅ |
| H | OAc | H | H | H | CH₃ & C₂H₅ |
| H | H | OAc | H | H | CH₃ & C₂H₅ |

16. A pharmaceutical composition comprising a compound of formula 1 as defined in claim 14 in an appropriate amount in an admixture with pharmaceutically acceptable carrier.
